(19) United States
(12) Patent Application Publication
(10) Pub. No.: US 2025/0312259 A1
(43) Pub. Date: Oct. 9, 2025

(54) SKIN CARE COMPOSITION COMPRISING WATER-SOLUBLE/DISPERSIBLE UV-BLOCKING AGENT, WAX AND PARTICULATE UV FILTER

(71) Applicant: The Procter & Gamble Company, Cincinnati, OH (US)

(72) Inventors: Lu Zhang, Singapore (SG); Eileen Yu Min Chew, Singapore (SG)

(21) Appl. No.: 19/172,743

(22) Filed: Apr. 8, 2025

Related U.S. Application Data
(60) Provisional application No. 63/575,900, filed on Apr. 8, 2024.

(51) Int. Cl. A61K 8/49; A61K 8/92; A61Q 17/04

(52) U.S. Cl. CPC A61K 8/4946; A61K 8/496; A61K 8/92; A61Q 17/04; A61K 2800/412

(57) ABSTRACT

A skin care composition that includes from about 0.01% to about 50% by weight of the composition, of water-soluble or water-dispersible UV-blocking active, from about 0.1% to about 50% by weight of the composition, of a wax having a melting point of from about 68° C. to about 95° C. and having an average particle size in the composition of from about 100 nm to about 100 microns, and from about 0.1% to about 20% by weight of the composition, of a particulate UV filter. The skin care compositions can provide improved UV protection, while providing good skin feel.

### FIELD OF THE INVENTION

The present disclosure generally relates to a skin care composition, comprising: from about 0.01% to about 50% by weight of the composition, of water-soluble or water-dispersible UV-blocking active; from about 0.1% to about 50% by weight of the composition, of a wax having a melting point of from about 68° C. to about 95° C. and having an average particle size in the composition of from about 100 nm to about 100 microns; and from about 0.1% to about 20% by weight of the composition, of a particulate UV filter. Such skin care compositions provide improved UV protection, while providing good skin feel.

### BACKGROUND OF THE INVENTION

It is well known that UV radiation (that is, wavelengths ranging from 280 nm to 400 nm) can cause damages to the human epidermis. More particularly, UV rays with wavelengths ranging from 320 to 400 nm (known as UV-A) may cause hyperpigmentation of the skin and long-term damages therein (such as wrinkles and fine lines), and rays with wavelength ranging from 280 to 320 nm (known as UV-B) can lead to erythemas and skin burns. In order to prevent or minimize damages or harmful effects caused by UV radiation, skin care compositions containing UV-blocking compounds in varying concentrations (also called sunscreen compositions) are provided.

Generally, in order to achieve higher UV protection, higher levels of oil-soluble or oil-dispersible UV blocking agents are used. For example, Lubrizol Advanced Material's WO 2021076474A1 is directed to a water-resistant and/or photoprotective composition comprising: a) an aqueous phase comprising from 0% to 99.9% by weight of the composition, based on the total weight of the composition; b) an oil phase comprising from 0% to 99.9% by weight of the composition, based on the total weight of the composition; c) an active sun block agent; and d) a micronized, non-solubilized wax comprising 0.1% to 10% by weight of the composition, based on the total weight of the composition; wherein the wax has a D50 particle size of 1 to 100 micro meters, and a melting point of at least 70° C.; and wherein the composition comprises at least 50% by weight of at least one of the aqueous phase or the oil phase. Such compositions are said to provide improved SPF and/or water-resistance by incorporating micronized, non-soluble waxes into the compositions. Lubrizol Advanced Material's WO 2021076474A1 discloses, for example, a composition in Example 157 in Table 26 in paragraph [174] and paragraph [176] containing: total 28% of oil-soluble UV blocking agents (10% octocrylene, 10% homosalate, 5% ethylhexyl salicylate, 3% butyl methoxydibenzoyl methane); 5% Wax described as the SPF booster which is non-solubilized and which is a mixture of carnauba was and rice bran wax; and 49.15% water.

However, such compositions containing higher levels of oil-soluble or oil-dispersible UV blocking agents tend to provide oily/greasy feel. This oily/greasy feel may discourage consumers to use adequate amount of the product. Also, sunscreen compositions are more frequently used during warmer seasons, such as in the summer, many consumers desire fresh feel and/or reduced oily/greasy feel to the skin upon use of sunscreen compositions.

Another way to achieve higher UV protection is to use higher levels of inorganic particulate UV blocking agents such as titanium dioxide and zinc oxide. For example, Shiseido's EP4079286A1 is directed to a sunscreen cosmetic containing: (A) an oil phase thickener; (B) an ultraviolet protectant; and (C) a wax powder having a volume-average particle size of 1 to 30 μm. Shiseido's EP4079286A1 said that the objective is to provide a sunscreen cosmetic that, by blending an environmentally friendly and highly water-resistant powder component instead of microplastic beads or silica, can achieve high ultraviolet protection power even when a small amount of an ultraviolet protectant is used. Shiseido's EP4079286A1 discloses a composition in Example 3 containing: total 13.3% of oil soluble UV blocking agents (5% octocrylene, 1% bis ethyhexyloxyphenol methoxyphenyl triazine, 2% diethylamino hydroxybenzonyl hexyl benzoate, 5% ethylhexyl salicylate, 0.3% dimethyldistearyl ammonium hectorine); total 13% of inorganic particulate UV blocking agents (2% titanium oxide, 6% zinc oxide, 5% another zinc oxide); 8% Wax powder B which is Carnauba/rice bran wax mixture with volume-average particle size of 4 to 14 μm; and 17.7% water.

However, such compositions containing higher levels of inorganic particulate UV blocking agents tend to provide negative sensory feel such as draggy feel and/or tend to leave undesired white residues on skin. Also, compositions containing reduced level of water and/or compositions in a form of water-in-oil emulsion may provide greasy feel and/or less fresh feel.

Accordingly, there remains a need for a UV-blocking composition which provides not only effective UV protection, but also a good skin feel (for example, fresh feel, reduced oily/greasy feel, reduced draggy feel, and/or reduced white residues).

### SUMMARY OF THE INVENTION

Disclosed herein is a skin care composition, comprising: from about 0.01% to about 50% by weight of the composition, of water-soluble or water-dispersible UV-blocking active; from about 0.1% to about 50% by weight of the composition, of a wax having a melting point of from about 68° C. to about 95° C. and having an average particle size in the composition of from about 100 nm to about 100 microns; and from about 0.1% to about 20% by weight of the composition, of a particulate UV filter. Such skin care compositions provide improved UV protection, while providing good skin feel (for example, fresh feel, reduced oily/greasy feel, reduced draggy feel, and/or reduced white residues).

It has been surprisingly found that, by using the combination of the water-soluble/dispersible UV blocking agent, the particulate wax, and the particulate UV filter especially the organic particulate UV filter such as Methylene Bis-Benzotriazolyl Tetramethylbutylphenol, the composition significantly improves UV protection, while providing good skin feel. In other words, by the use of the above combination, the composition can achieve improved UV protection, without the need for too high level of the oil-soluble/dispersible UV 5 blocking agents and/or without the need for too high level of inorganic particulate UV filters.

Preferably, by the use of oil-soluble/dispersible UV blocking agents together with the above combination, the composition further improves UV protection.

Also, by the use of the above ingredients at preferred concentrations and/or at preferred ratios, the composition further improves UV protection and/or sensory to skin.

### DETAILED DESCRIPTION OF THE INVENTION

As used in the description and the appended claims, the singular forms “a,” “an,” and “the” are intended to include the plural forms as well, unless the context clearly indicates otherwise. All percentages and ratios used herein are by weight of the total composition, unless otherwise designated. The number of significant digits conveys neither a limitation on the indicated amounts nor on the accuracy of the measurements. All numerical amounts are understood to be modified by the word “about” unless otherwise specifically indicated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Definitions

“Apply” or “application,” as used in reference to a composition, means to apply or spread the composition onto keratinous tissue such as the epidermis.

“Keratinous tissue” refers to keratin-containing layers disposed as the outermost protective covering of mammals (e.g., humans, dogs, cats, etc.) which includes, but is not limited to, skin, lips, hair, toenails, fingernails, cuticles, hooves, etc.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“UV-blocking active” means a material recognized by a skilled artisan in the field of sunscreen formulation to be a dermatologically acceptable UV-absorbing or UV-reflecting active material. Such UV-blocking actives may be described as being UVA-blocking and/or UVB-blocking active agents. Approval by a regulatory agency is generally required for inclusion of active agents in formulations intended for human use. Those active agents which have been or are currently (per 21 C.F.R. part 352) approved by the U.S. Food and Drug Administration as acceptable for use in over-the-counter sunscreen drug products include organic and inorganic substances including, without limitation, para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the U.S., but are approved for over-the-counter use in other regions or countries such as Europe (per European Commission's Cosmetic Directive Regulation), Japan, China, Australia, New Zealand, or Canada include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. However, as the list of approved materials is currently expanding, those of ordinary skill will recognize that the composition is not limited to UV-blocking actives currently approved for human use but are readily applicable to those that may be allowed in the future.

“Skin care composition” refers to topical personal care compositions for regulating and/or improving a skin condition. Some nonlimiting examples of skin care compositions include solutions, aerosols, liposomes, creams, moisturizers, gels, lotions, ointments, serums, sticks, spray, emulsions, or any other dispersions, for example facial cleansers, body washes, a sunscreen cream, a sunscreen spray, a sunscreen emulsion, a skincare base, a makeup base, a tinted cream, a foundation in liquid, an eye contour care product, a concealing care serum, a concealer stick or a natural makeup product for the lips, a liquid gloss, a lipstick paste, and the like.

“Topical” refers to a composition that is intended to be applied to a keratinous surface such as skin or hair.

“Derivatives” means an ester, ether, amide, hydroxy, and/or salt structural analogue of the relevant compound.

### Skin Care Composition

Skin care composition herein comprises: from about 0.01% to about 50% by weight of the composition, of water-soluble or water dispersible UV-blocking active; from about 0.1% to about 50% by weight of the composition, of a wax having a melting point of from about 68° C. to about 95° C. and having an average particle size in the composition of from about 100 nm to about 100 microns; and from about 0.1% to about 20% by weight of the composition, of a particulate UV filter.

Such skin care compositions provide improved UV protection, while providing good skin feel.

The skin care compositions herein are configured for topical application to keratinous tissue.

### Water-Soluble or Water-Dispersible UV-Blocking Active

The skin care composition comprises a water-soluble or water-dispersible UV-blocking active. The skin care composition according to claim any one of the preceding claims, wherein the composition comprises from about 0.01% to about 50%, preferably from about 0.1% to about 30%, more preferably from about 0.5% to about 10%, most preferably from about 1% to about 5%, by weight of the composition, of said water-soluble/dispersible UV-blocking active.

The water-soluble/dispersible UV-blocking active useful herein is preferably selected from the group consisting of phenylbenzimidazole sulfonic acid, salts thereof, terephthalylidene dicamphor sulfonic acid, salt thereof, disodium phenyl dibenzimidazole tetrasulfonate and any combinations thereof.

More preferably, said water-soluble/dispersible UV-blocking active is selected from the group consisting of phenylbenzimidazole sulfonic acid salts thereof, and any combinations thereof.

In the composition, water-soluble UV blocking agent is preferably used compared to water-dispersible UV blocking agent, in view of better/even distribution in water phase and better coverage of skin. It is known that oil soluble UV blocking actives can be surface modified/coated to be water-dispersible. One such example is a special grade of Bis-Ethylhexyloxyphenol Methoxyphenyl Triazine, market by BASF under trade name of Tinosorb S lite Aqua. However, it is preferred to use water-soluble UV-blocking actives which are water-soluble as nature and/or without surface modification/coating, in view of improved sensory feel, better/even distribution in water phase and better coverage of skin.

### Wax

The composition herein comprises a wax. The wax is included in the composition at a level by weight of the composition, from about 0.1% to about 50%, preferably from about 0.3% to about 30%, more preferably from about 0.5% to about 20%, still more preferably from about 0.5 to about 5%.

The wax useful herein has a melting point of from about 68° C. to about 95° C., preferably from about 72° C. to about 90° C. The wax useful herein has a particle size in the composition of from about 100 nm to about 100 microns, preferably from about 400 nm to about 50 microns, more preferably from about 1 microns to about 20 microns, in view of providing improved UV protection especially when combining with water-soluble/dispersible UV blocking agent. If the wax has lower melting point, the wax may not be able to have the above particle size in the composition, as the wax may melt and/or be mixed homogeneously with other ingredients during manufacturing of the composition. Particle size herein is an average particle size measured by a particle size analyzer named MasterSizer 3000 available from Malvern which, is based on laser diffraction.

Commercially available examples of such wax include, for example, Sunhancer available from Lubrizol: which is a mixture of Oryza Sativa (Rice) Bran Wax having a melting point of 77-82° C. and Carnauba wax having a melting point of 82-86° C., with the mixture melting point of 78-86° C.; which has an average particle size of 6.5-7.5 micrometer and particle size distribution Dv(10)-(90): 4-16 μm.

### Particulate UV Filter

The composition comprises a particulate UV filter. The particulate UV filter is included in the composition at a level by weight of the composition, from about 0.1% to 20%, preferably from about 1% to about 10%, more preferably from about 1% to about 8%.

The particulate UV filter is selected from the group consisting of: organic particulate UV filters such as Methylene Bis-Benzotriazolyl Tetramethylbutylphenol (such as Tinosorb M), Tris Biphenyl Triazine (marketed by BASF under trade name of Tinosorb A2B), Bis(Diethylaminohydroxybenzoyl Benzoyl) Piperazine (marketed by BASF under the trade name of HAA299); inorganic particulate UV filters such as TiO₂, ZnO; and mixtures thereof.

Among inorganic particulate UV filters, TiO₂ is usually preferred over ZnO in view of its better efficacy in providing SPF protection. When the inorganic particulate UV filters are included, the level of the inorganic particulate UV filters is preferably up to about 10%, more preferably up to about 8%, still more preferably up to about 5%.

Among organic and inorganic particulate UV filters, preferred are organic particulate UV filters in view of the improved UV protection and sensory feel, and more preferred is Methylene Bis-Benzotriazolyl Tetramethylbutylphenol supplied from BASF as Tinosorb® M. This preferred UV filter stays in a water phase together with the water-soluble/dispersible UV blocking agent while the preferred wax stays in an oil phase, and provide synergistic improvement in UV protection together with the water-soluble/dispersible UV blocking agent and wax in composition containing both water phase and oil phase, especially oil-in-water emulsion containing a continuous water phase and dispersed oil phases.

### Oil-Soluble or Oil Dispersible UV-Blocking Active

The skin care composition preferably further comprises an oil-soluble or oil dispersible UV-blocking active. The composition comprises preferably from about 0.01% to about 50%, more preferably from about 0.1% to about 40%, still more preferably from about 1% to about 30%, even more preferably from about 5% to about 25%, most preferably from about 13% to about 23%, by weight of the composition, of said oil-soluble/dispersible UV-blocking active.

The oil-soluble/dispersible UV-blocking active is preferably selected from the group consisting of homosalate, octocrylene, octisalate, butyl methoxydibenzoylmethane, diethylamino hydroxybenzoyl hexyl benzoate, bis-ethyl-hexyloxyphenol methoxyphenyl triazine, ethylhexyl methoxycinnamate, ethylhexyl triazone (commercially available as Uvinul® T 150 from BASF), drometrizole trisiloxane (marketed as Mexoryl XL by L'Oreal), and any combinations thereof.

Among them, preferred oil-soluble/dispersible UV-blocking active is selected from the group consisting of homosalate, octocrylene, octisalate, butyl methoxydibenzoylmethane, diethylamino hydroxybenzoyl hexyl benzoate, bis-ethyl-hexyloxyphenol methoxyphenyl triazine, ethylhexyl methoxycinnamate, and any combinations thereof. More preferred oil-soluble/dispersible UV-blocking active is selected from the group consisting of Homosalate, Octocrylene, Octisalate, butyl methoxydibenzoylmethane. and any combinations thereof. In the composition, oil-soluble UV blocking agent is preferably used compared to oil-dispersible UV blocking agent, in view of better/even distribution in oil phase and better coverage of skin.

The composition preferably comprises from about 10% to about 60%, more preferably from about 15% to about 55%, still more preferably from about 17% to about 50%, even more preferably from about 20% to about 45%, by weight of the composition, of a sum of the water-soluble/dispersible UV-blocking active and the oil-soluble/dispersible UV-blocking active.

### Ratio Between Water-Soluble/Dispersible UV Blocking Active and Particulate UV Filter.

Without intending to be bound by theory, it is believed that the combined use of a preferred concentration of the water-soluble/dispersible UV-blocking active and a preferred concentration of the particulate UV filter at preferred ratios brings additional benefits, such as improved UV protection and/or even better sensory benefits. Such preferred ratio between the water-soluble/dispersible UV-blocking active and the particulate UV filter is from about 3.5:1 to about 1:10, more preferably from about 3:1 to about 1:5, still more preferably from about 2:1 to about 1:2.

### Ratio Between Water-Soluble/Dispersible UV Blocking Active and Oil-Soluble/Dispersible UV-Blocking Active.

Without intending to be bound by theory, it is believed that the combined use of a preferred concentration of the water-soluble/dispersible UV-blocking active and a preferred concentration of the oil soluble/dispersible UV-blocking active at preferred ratios brings additional benefits, such as even better sensory benefits. Such preferred ratio between the water-soluble/dispersible UV-blocking active and the oil soluble/dispersible UV-blocking active is from about 1:1 to about 1:25, more preferably from about 1:2 to about 1:10, still more preferably from about 1:3 to about 1:7.

### Aqueous Carrier/Water

The composition preferably comprises an aqueous carrier. Preferably, the aqueous carrier is substantially water. Deionized water is preferably used. Water from natural sources including mineral cations can also be used, depending on the desired characteristic of the composition.

The composition comprises aqueous carrier/water at a level of from about 30% to about 99%, preferably from about 40% to about 95%, more preferably from about 45% to about 90%, still more preferably from about 50% to about 85%, even more preferably from about 50% to about 80%, by weight of the composition.

In some instances, the compositions herein are in the form of an oil-in-water (“O/W”) emulsion that provides a sensorial feel that is fresh, light and/or non-greasy. Suitable O/W emulsions herein may include a continuous aqueous phase of more than 50% by weight of the composition, and the remainder being a dispersed oil phase.

### Dimethicone

The present composition may comprise from about 2% to about 45%, preferably from about 3% to about 40%, more preferably from about 4% to about 30%, yet more preferably from about 4.5% to about 20%, and most preferably from about 4.5% to about 10%, for example about 3%, about 3.5%, about 4%, about 4.5%, about 5%, about 5.5%, about 6%, about 6.5%, about 7%, about 7.5%, about 8%, about 8.5%, about 9%, about 9.5%, about 10% or any ranges therebetween, by weight of the composition, of dimethicone. Particularly, the dimethicone may be a single dimethicone or a blend of two or more dimethicones disposed in the oil phase of the composition. Preferably, dimethicone in the composition of the present disclosure is a low-viscosity dimethicone. In some embodiments, dimethicone in the composition of the present disclosure is non-volatile.

As used herein, the term of “dimethicone” means a polydimethylsiloxane compound having the formula:

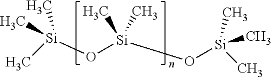

Dimethicone may be manufactured in multiple viscosities depending on the molecular mass, ranging from around 0.5 cSt to 20 million cSt. Particularly, dimethicone is in the form of a thin pourable liquid when the molecular mass is very low, and in the form of a thick rubbery semi-solid when the molecular mass is very high. Low-viscosity dimethicone are commonly used in skin care compositions as carriers for delivering a wide range of ingredients to skin without leaving a heavy, greasy feel. However, it has also some disadvantages, for example such low-viscosity dimethicone may be perceived as dry. As used herein, the term of “low-viscosity dimethicone” means a dimethicone having a relatively low kinematic viscosity, preferably no more than about 1000 cSt, and more preferably no more than about 500 cSt.

It was surprisingly discovered by the present inventors that a high SPF UV composition with an unexpectedly well-balanced skin feel could be formed by using a low-viscosity dimethicone in combination with other ingredients such as particulate materials.

The kinematic viscosity of the dimethicone which is useful in the composition of the present disclosure may be within the range of from about 0.5 cSt to about 1000 cSt, preferably from about 1 cSt to about 500 cSt, more preferably from about 1.5 cSt to about 350 cSt, yet more preferably from about 2 cSt to about 200 cSt, yet more preferably from about 3.5 cSt to about 100 cSt, yet more preferably from about 3.5 cSt to about 50 cSt, and most preferably from about 3.5 cSt to about 20 cSt, for example about 0.5 cSt, about 0.65 cSt, about 1 cSt, about 1.5 cSt, about 2 cSt, about 2.5 cSt, about 3 cSt, about 3.5 cSt, about 4 cSt, about 4.5 cSt, about 5 cSt, about 6 cSt, about 7 cSt, about 8 cSt, about 9 cSt, about 10 cSt, about 12 cSt, about 15 cSt, about 18 cSt, about 20 cSt, about 30 cSt, about 40 cSt, about 50 cSt, about 100 cSt, about 200 cSt, about 300 cSt, about 400 cSt, about 500 cSt or any ranges therebetween, as measured by the Rheology Method. Without intending to be bound by theory, it is believed that preferred concentrations of dimethicone and/or preferred kinematic viscosity of dimethicone may bring about additional benefits, such as higher stability, even better sensory benefits, suitable moisturization perception and/or product aesthetics. More particularly, it may help to stabilize the formula when exposed to low temperature conditions and prevents destabilization caused by other ingredients (for example the particulate material).

Particularly useful low-viscosity dimethicone include Andisil® SF 5, Andisil® SF 50, Andisil® SF 500, Andisil® SF 1000, Clearco PSF 0.65 cSt, PSF 1.5 cSt, PSF 2 cSt, Dow Corning 200® Fluid 0.65cs, Dow Corning 200® Fluid 1.5cs, Dow Corning Xiameter® PMX-200 silicone fluid 5 cst, Dow Corning 200® Fluid 10 cSt, GE SF96-5, GE SF96-10cs, GE SF96-20cs, Momentive SF96-5, Momentive SF96-10, Momentive SF96-20, Rhodorsil 47V5, Rhodorsil 47V10, Rhodorsil 47V20, Shinetsu DM5, Shinetsu DM10, Shinetsu DM20.

Particulate Material (Other than Wax or Particulate UV Filter)

The skin care composition may comprise from 3.5% to 40%, preferably from 4% to 30%, more preferably from 4.5% to 20%, most preferably from 4.5% to 10%, by weight of the composition, of said particulate material; wherein preferably said particulate material is selected from the group consisting of sugar derivatives, silica, silicates, carbonates, silicones and any mixtures thereof.

The compositions may comprise from about 3% to about 45%, preferably from about 3.5% to about 40%, more preferably from about 4% to about 30%, yet more preferably from about 4.5% to about 20%, and most preferably from about 4.5% to about 10%, for example about 3%, about 3.5%, about 4%, about 4.5%, about 5%, about 5.5%, about 6%, about 6.5%, about 7%, about 7.5%, about 8%, about 8.5%, about 9%, about 9.5%, about 10% or any ranges therebetween, by weight of the composition, of a particulate material (also called cosmetic powder or dry powder).

Non-limiting examples of suitable particulate materials include inorganic powders, organic powders, composite powders, optical brightener particles, and mixtures of any of the foregoing. These particulates can, for instance, be platelet shaped, spherical, elongated or needle-shaped, or irregularly shaped; surface coated or uncoated; porous or non-porous; charged or uncharged. In some embodiments, the particulate material is hydrophobically coated.

Suitable inorganic particulate materials include, but are not limited, to talc, silicates, silica, iron oxides, and any mixtures thereof.

Suitable organic particulate materials include, but are not limited, to polymeric particles chosen from the methylsilsesquioxane resin microspheres, for example, Tospearl™ 145A, Tospearl™ CF600; microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); spherical particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (Akzo Nobel); starch for example tapioca starch, distarch phosphate, tapioca starch, acetyl substituted starch, adipic acid substituted starch, hydroxypropylated starch, corn starch, 2-hydropropylether modified starch, hydroxypropyl starch phosphate ester; cyclodextrins and derivatives thereof; polymethyl silsesquioxane coated tapioca particles, for example Dry Flo TS™ (Akzo Nobel); microspheres of polyethylene, for example Microthene™ FN510-00 (Equistar), silicone resin, polymethylsilsesquioxane silicone polymer, platelet shaped powder made from L-lauroyl lysine, and mixtures thereof.

Particularly, the particulate material may be selected from the group consisting of sugar derivatives, silica, silicates, carbonates, silicones and any mixtures thereof. More particularly, the sugar derivatives may be selected from the group consisting of aluminium starch octenylsuccinate, distarch phosphate, tapioca starch, acetyl substituted starch, adipic acid substituted starch, hydroxypropylated starch, corn starch, 2-hydropropylether modified starch, hydroxypropyl starch phosphate ester, cyclodextrins and derivatives thereof, polymethylsilsesquioxane-coated starch such as polymethylsilsesquioxane-coated tapioca starch, and any combinations thereof; and/or the silicates may be selected from talc, kaolin and a combination thereof; and/or the carbonates may be selected from magnesium carbonate, calcium carbonate and a combination thereof; and/or the silicone may be are selected from vinyl dimethicone/methicone silsesquioxane crosspolymer, polysilicone, polymethylsilsesquioxane, diphenyl dimethicone/vinyl diphenyl dimethicone/silsesquioxane crosspolymer, and any combinations thereof.

In some embodiments, the average diameter of the longest side of the individual particles of particulate materials is within the range of between about 1 micron and about 150 microns, preferably less than about 75 microns, and more preferably less than about 50 microns, for example about 1 micron, about 2 micron, about 3 micron, about 4 micron, about 5 micron, about 1 micron, about 10 micron, about 15 micron, about 30 micron, or any ranges therebetween.

Weight Ratio Between Low-Viscosity Dimethicone and Particulate Material (Other than Wax or Particulate UV Filter)

In some embodiments when containing the Low-Viscosity Dimethicone and the Particulate Material, it may be preferred that the weight ratio of the dimethicone to the particulate material in the skin care composition is between about 1:2 and about 2:1, preferably between about 1:1.5 and about 1.5:1, more preferably between about 1:1.2 and about 1.2:1, and most preferably between 1:1.1 and 1.1:1, for example about 1:2, about 1:1.8, about 1:1.5, about 1:1.3, about 1:1.2, about 1:1.1, about 1:1.05, about 1:1, about 1.05:1, about 1.1:1, about 1.2:1, about 1.3:1, about 1.5:1, about 1.8:1, about 2:1, or any ranges therebetween.

Without intending to be bound by theory, it is believed that preferred concentrations and/or preferred types of dimethicone (for example dimethicone, 5 cSt) and particulate materials (for example polymethylsilsesquioxane or polymethylsilsesquioxane-coated starch) and/or preferred ratio of dimethicone to particulate materials (for example between 1:1.1 and 1.1:1) may bring about additional benefits, such as higher stability, even better sensory benefits, suitable moisturization perception and/or product aesthetics.

### Fatty Alcohol

The compositions of the present disclosure may comprise one or more fatty alcohols. Fatty alcohols typically include monohydric alcohols having 8-22 carbon atoms although longer chain alcohols in excess of 30 carbons may be used. The fatty alcohols may be saturated or unsaturated. The fatty alcohols may be straight or branched. In particular, the compositions may comprise straight chain, saturated fatty alcohol with a terminal hydroxyl. Suitable fatty alcohols include decyl alcohol, lauryl alcohol, myristyl alcohol, cetyl alcohol, stearyl alcohol, isostearyl alcohol, cetearyl alcohol, icosyl alcohol, behenyl alcohol and the like. The phase may comprise from about 0.1% to about 5%, from about 0.5% to about 7.5%, from about 1% to about 10%, from about 3% to about 15%, from about 5% to about 20% of fatty alcohol.

Alternatively, the compositions of the present disclosure may be essentially free of one or more or any fatty alcohols. Particularly, the compositions of the present disclosure may be essentially free of a fatty alcohol selected from the group consisting of cetyl alcohol, stearyl alcohol, cetearyl alcohol, behenyl alcohol, and any combinations thereof.

In the context of the present disclosure, the expression of “essentially free of” means that the concentration, by weight of the composition, of an ingredient is no more than about 1%, preferably about 0.8%, more preferably about 0.5%, yet more preferably about 0.3%, yet more preferably about 0.2%, yet more preferably about 0.1%, and most preferably about 0.01%.

In some embodiments, the composition comprises from 0% to about 0.5%, preferably from 0% to about 0.2%, yet more preferably from 0% to about 0.1%, yet more preferably from 0% to about 0.05%, and most preferably from 0% to about 0.01%, for example 0%, about 0.01%, about 0.05%, about 0.1%, about 0.2%, about 0.3%, about 0.4%, about 0.5% or any ranges therebetween, by weight of the composition, of a fatty alcohol.

Without intending to be bound by theory, it is believed that the absence of fatty alcohols in the skin care composition may bring about additional benefits, such as higher stability, even better sensory benefits, suitable moisturization perception and/or product aesthetics.

### Emulsifier

The compositions of the present disclosure may further comprise from about 0.01% to about 40%, by weight of the composition, of an emulsifier. Emulsifiers may be nonionic, anionic or cationic.

Suitable emulsifiers may be selected from the group consisting of ether-based emulsifiers, ester-based emulsifiers, sugar based emulsifiers, polymeric emulsifiers, silicone emulsifiers, and any combinations thereof.

The ether-based emulsifiers may be selected from the group consisting of ethers of polyglycols and of fatty alcohols, ethers of polyglycols and of glycosylated fatty alcohols, ethers of C₁₂₋₃₀ alcohols and of glycerol or polyglycerol, ethers of oxyalkylene-modified C₁₂₋₃₀ alcohols and of glycerol or polyglycerol, ethers of C₁₂₋₃₀ fatty alcohols and of sucrose or glucose, ethers of sorbitol and/or sorbitan and of alkoxylated sorbitan, ethers of polyglycols and of cholesterol, and any combinations thereof.

The ester-based emulsifiers may be selected from the group consisting esters of polyglycols and of fatty acids, esters of polyglycols and of glycosylated fatty acids, esters of C₁₂₋₃₀ fatty acids and of glycerol or polyglycerol, esters of sucrose or glucose and of C₁₂₋₃₀ fatty acids, esters of pentaerythritol and of C₁₂₋₃₀ fatty acids, esters of sorbitol and/or sorbitan and of C₁₂₋₃₀ fatty acids, esters of C₁₂₋₃₀ fatty acids and of alkoxylated ethers of sorbitol and/or sorbitan, and any combinations thereof;

Nonlimiting examples of emulsifiers for use herein may include: polyethylene glycol 20 sorbitan monolaulrate (polysorbate 20), steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate. PPG-2 methyl glucose ether distearate, ceteth-10, diethanolamine cetyl phosphate, glyceryl stearate, PEG 40 hydrogenated castor oil, PEG-60 hydrogenated castor oil, Glycereth-25 PCA Isostearate, and mixtures thereof.

Sugar based emulsifiers like Emulgade® PL 68/50, which is a mix of Cetearyl Glucoside and Cetearyl Alcohol.

Silicone emulsifiers may be useful. Linear or branched type silicone emulsifiers may also be used. Particularly useful silicone emulsifiers include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin Etsu.

Polymeric emulsifiers may also be used. Preferably, the composition may comprise from about 0.02% to about 30%, from about 0.03% to about 20%, from about 0.05% to about 5%, more preferably from about 0.1% to about 2%, yet more preferably from about 0.2% to about 1%, and most preferably from about 0.3% to about 0.8%, for example, about 0.3%, about 0.35%, about 0.4%, about 0.45%, about 0.5%, about 0.55%, about 0.6%, about 0.65%, about 0.7%, about 0.75%, about 0.8% or any ranges therebetween, by weight of the composition, of a polymeric emulsifier.

Preferred polymeric emulsifier may be selected from the group consisting of crosslinked alkyl acrylates, acryloyldimethyltaurate polymers, and any combinations thereof. Crosslinked alkyl acrylates are crosslinked polymers in which the co-monomers consist of at least one of the following: acrylic acid, sodium acrylate, methacrylic acid, or alkyl acrylate, and acryloyldimethyltaurate polymers mean homopolymers, copolymers or crosslinked polymers, the monomers of which are at least partially composed of acryloyldimethyltaurate monomers.

Particularly, the crosslinked alkyl acrylates are selected from the group consisting of acrylates/C10-30 alkyl acrylate crosspolymer, acrylates/C12-13 alkyl methacrylates/methoxyethyl acrylate crosspolymer, acrylates crosspolymer, acrylates/ethylhexyl acrylate crosspolymer, acrylates/ethylhexyl acrylate/glycidyl methacrylate crosspolymer, acrylates/PEG-4 dimethacrylate crosspolymer, acrylates/Steareth-20 methacrylate crosspolymer, acrylates/vinyl isodecanoate crosspolymer, acrylates/vinyl neodecanoate crosspolymer, allyl methacrylate/glycol dimethacrylate crosspolymer, allyl methacrylates crosspolymer, butyl acrylate/glycol dimethacrylate crosspolymer, C8-22 alkyl acrylates/methacrylic acid crosspolymer, glycol dimethacrylate/vinyl alcohol crosspolymer, lauryl methacrylate/glycol dimethacrylate crosspolymer, lauryl methacrylate/sodium methacrylate crosspolymer, methacrylic acid/PEG-6 methacrylate/PEG-6 dimethacrylate crosspolymer, PEG/PPG-5/2 methacrylate/methacrylic acid crosspolymer, stearyl/lauryl methacrylate crosspolymer, and any combinations thereof; and/or the acryloyldimethyltaurate polymers may be selected from the group consisting of acrylamide/Sodium acryloyldimethyltaurate copolymer, acrylamide/sodium acryloyldimethyltaurate/acrylic acid copolymer, ammonium acryloyldimethyltaurate/beheneth-25 methacrylate crosspolymer, ammonium acryloyldimethyltaurate/carboxyethyl, acrylate crosspolymer, ammonium acryloyldimethyltaurate/Laureth-7 methacrylate copolymer, ammonium acryloyldimethyltaurate/Steareth-25 methacrylate crosspolymer, ammonium acryloyldimethyltaurate/Steareth-8 methacrylate copolymer, ammonium acryloyldimethyltaurate/vinyl formamide copolymer, ammonium acryloyldimethyltaurate/VP copolymer, ammonium polyacryloyldimethyl taurate dimethylacrylamide/sodium acryloyldimethyltaurate crosspolymer, HEA/sodium acryloyldimethyltaurate/Steareth-20 methacrylate copolymer, hydroxyethyl acrylate/sodium acryloyldimethyl taurate copolymer, polyacryloyldimethyltaurate polyoxymethylene melamine, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acryloyl dimethyl taurate/PEG-8 diacrylate crosspolymer, sodium acryloyldimethyl taurate/acrylamide/VP copolymer, sodium acryloyldimethyltaurate/methacrylamidolauric acid copolymer, sodium acryloyldimethyltaurate/VP crosspolymer, sodium polyacryloyldimethyl taurate and any combinations thereof.

Non-limiting examples of polymeric emulsifiers are Carbopol 1382™, Carbopol Ultrez 20™, Carbopol Ultrez 21™, Pemulen TR II™, Pemulen TR I™, Pemulen EZ4U™, Aculyn 88, Aculyn 38, Stabylen 30, Aqua Keep 10SH-NFC, Aristoflex® AVC, SEPINOV™ EMT 10, SIMULGEL™ NS, SEPIPLUS™ S, EMT polymer, Aristoflex® Silk Polymer, Aristoflex® Eco T, PemuPur™ and the like.

In some preferred embodiments, the composition of the present disclosure comprises acrylates/C10-30 alkyl acrylate crosspolymer and/or hydroxyethyl acrylate (and) sodium acryloyldimethyl (and) taurate copolymer. The use of such polymeric emulsifiers surprisingly provides additional sensory benefits. Without intending to be bound by theory, it is believed that they function also as thickeners to prevent use of other thickeners/emulsifiers that may negatively impact on the sensory perception of the product, and in other words, they can emulsify oil and water and also thicken the composition by the use of a single material.

Without intending to be bound by theory, it is believed that that preferred concentrations and/or preferred types of the emulsifier bring about additional benefits, such as higher stability, even better sensory benefits, suitable moisturization perception, more cost-effective and/or product aesthetics.

### PH Adjuster

The composition of the present disclosure may further comprise a pH adjuster to control the pH of the composition. Particularly, the pH of the composition of the present disclosure is within the range of from about 5 to about 8, preferably from about 5.2 to about 7.8, and more preferably from about 5.4 to about 7.6, for example about 5.4, about 5.6, about 5.8, about 6.0, about 6.2, about 6.4, about 6.6, about 6.8, about 7.0, about 7.2, about 7.4, about 7.6 and any ranges therebetween.

Particularly, the composition may further comprise from about 0.01% to about 5%, preferably from about 0.1% to about 3%, more preferably from about 0.3% to about 2%, yet more preferably from about 0.4% to about 1.8%, and most preferably from about 0.5% to about 1.6%, for example about 0.4%, about 0.5%, about 0.6%, about 0.7%, about 0.8%, about 0.9%, about 1.0%, about 1.1%, about 1.2%, about 1.3%, about 1.4%, about 1.5%, about 1.6% and any ranges therebetween, by weight of the composition, of a pH adjuster, wherein said pH adjuster is selected from the group consisting of potassium hydroxide, sodium hydroxide, ammonium hydroxide, aminomethyl propanol, triethanolamine, tetrahydroxypropyl ethylene diamine and any combinations thereof.

When a polymeric emulsifier and a pH adjuster is used, it is preferred, and sometimes necessary in the case of certain compositions, to formulate the composition of the present disclosure with the proper ratio of the polymeric emulsifier to the pH adjuster. In some embodiments, the weight ratio of the polymeric emulsifier to the pH adjuster is between about 1:5 and about 1:0.5, preferably between about 1:3 to about 1:1, for example about 1:3, about 1:2.5, about 1:2, about 1:1.5, about 1:1.1 and any ranges therebetween.

Without intending to be bound by theory, it is believed that that preferred concentrations and/or preferred types of the pH adjuster bring about a more preferred result, such as higher stability, even better sensory benefits, suitable moisturization perception, more cost-effective and/or product aesthetics.

### Thickener

The composition may further comprise a thickener (also called a thickening agent) or an additional thickener if the emulsifier in the composition also functions as a thickener. The composition may comprise from about 0.1% to about 5%, or, alternatively, from about 0.2% to about 2%, of a thickener or an additional thickener when present. Suitable classes of thickeners include but are not limited to carboxylic acid polymers, polyacrylamide polymers, sulfonated polymers, copolymers thereof, hydrophobically modified derivatives thereof, and mixtures thereof.

One preferred thickener is an acrylate cross linked silicone copolymer network (also sometimes referred to as “polyacrylate siloxane copolymer network”). Suitable thickening agents include carboxylic acid polymers, polyacrylamide polymers or copolymers, sulfonated polymers, gum, clays, cellulose or modified cellulosic compositions, and the like.

### Other Optional Ingredients

The present composition may include a variety of optional ingredients that are known for use in personal care composition, as long as the optional ingredient(s) do not unduly alter product stability, aesthetics, or performance. The optional ingredients, when incorporated into the composition, should be suitable for contact with human keratinous tissue without undue toxicity, incompatibility, instability, allergic response, and the like within the scope of sound judgment. The compositions herein may comprise from about 0.0001% to about 50%; from about 0.001% to about 20%; or, alternately, from about 0.01% to about 10%, by weight of the composition, of optional ingredients. Some non-limiting examples of optional ingredients include abrasives, absorbents, opacifying agents, colorings/colorants (e.g., pigments, dyes, and lakes), particles, essential oils, anti-caking agents, foaming agents, anti-foaming agents, oil control agents, binders, biological additives, vitamins, minerals, peptides, sugar amines, flavonoid compounds, anti-oxidants, preservatives, plant extract, phytosterols, protease inhibitors, tyrosinase inhibitors, exfoliating agents, skin lightening agents, sunless tanning agents, anti-acne actives, anti-cellulite actives, anti-wrinkle actives, phytosterols and/or plant hormones, N-acyl amino acid compounds, antimicrobials, antifungals, moisturizers, emollients, humectants, lubricating agents, fragrances, anti-dandruff agents, buffering agents, bulking agents, chelating agents, biocides, denaturants, astringents, external analgesics, anti-inflammatory agents, sunscreen agents, film formers and/or polymers for aiding film-forming properties and substantivity of the composition, propellants, reducing agents, sequestrants, conditioning agents, and combinations of these.

In some embodiments, the compositions of the present disclosure may be essentially free of ethanol. Without intending to be bound by theory, it is believed that that the absence of ethanol in the compositions of the present disclosure brings about a more preferred result, such as even better sensory benefits and/or product aesthetics.

### Combinations

1. A skin care composition, comprising:
   a) from about 0.01% to about 50% by weight of the composition, of water-soluble or water-dispersible UV-blocking active;
   b) from about 0.1% to about 50% by weight of the composition, of a wax having a melting point of from about 68° C. to about 95° C. and having an average particle size in the composition of from about 100 nm to about 100 microns; and
   c) from about 0.1% to about 20% by weight of the composition, of a particulate UV filter.
2. The skin care composition of the paragraph 1, wherein the particulate UV filter is selected from the group consisting of an organic particulate UV filter and an inorganic particulate UV filter, and mixtures thereof, preferably wherein the particulate UV filter is an organic particulate UV filter, more preferably wherein the organic particulate UV filter is Methylene Bis-Benzotriazolyl Tetramethylbutylphenol.
3. The skin care composition of any of the preceding paragraphs, comprising from about 1% to about 10% of the particulate UV filter, preferably from about 1% to about 8% of the particulate UV filter.
4. The skin care composition of any of the preceding paragraphs, wherein the weight ratio between the water-soluble/dispersible UV-blocking active and the particulate UV filter is from about 3.5:1 to about 1:10, preferably from about 3:1 to about 1:5, more preferably from about 2:1 to about 1:2.
5. The skin care composition according to any of the preceding paragraphs, wherein the composition further comprises from about 30% to about 99% of an aqueous carrier.
6. The skin care composition according to any of the preceding paragraphs, wherein the composition is in a form of an oil-in-water emulsion.
7. The skin care composition according to any of the preceding paragraphs, wherein the water-soluble/dispersible UV-blocking active is preferably selected from the group consisting of phenylbenzimidazole sulfonic acid, salts thereof, terephthalylidene dicamphor sulfonic acid, salt thereof, disodium phenyl dibenzimidazole tetrasulfonate and any combinations thereof, preferably wherein the water-soluble/dispersible UV-blocking active is selected from the group consisting of phenylbenzimidazole sulfonic acid salts thereof, and any combinations thereof.
8. The skin care composition according to any of the preceding paragraphs, comprising from about 0.5% to about 10%, by weight of the composition, of said water-soluble/dispersible UV-blocking active.
9. The skin care composition according to any of the preceding paragraphs, wherein the composition further comprises from about 5% to about 25%, preferably from about 13% to about 23%, by weight of the composition, of the oil-soluble/dispersible UV-blocking active.
10. The skin care composition according to any of the preceding paragraphs, wherein the weight ratio between the water-soluble/dispersible UV blocking active and the oil-soluble/dispersible UV-blocking active is from about 1:1 to about 1:25, preferably from about 1:2 to about 1:10, more preferably from about 1:3 to about 1:7.

### TEST METHODS

### In Vitro SPF Test Method

This in-vitro SPF method is based on UVR transmittance spectroscopy, whereby spectroradiometric measurement of UVR transmission through appropriate UV-transparent substrates (with and without applied sunscreen products), allows prediction of in vivo SPF values.

Using a positive-displacement pipette, 1.3 mg/cm2 of product is applied to each moulded plate (HD6) and 1.2 mg/cm2 of the same product is applied to each sandblasted plate (SB6). The product is applied uniformly to each plate by depositing at least 9 drops of the same volume to the same location, using a standard template as a guide.

Once the product is deposited in this manner, each plate is immediately placed on the stage of a robot (e.g., Spreadmaster from Helioescreen) with a product-saturated latex finger-cot placed on the application probe and the automated spreading sequence starts. After application of the product, the plates are kept in a darkened incubator for 30 min at 25° C. The absorbance spectrum (290-400 nm) of product films spread on either moulded or sandblasted plates is measured using a calibrated spectrophotometer (E.g., Labsphere 2000). A separate blank measurement shall also be performed on both moulded or sandblasted plates, spread with glycerine or white petroleum.

Absorbance data from both plates are then combined to yield a single term describing initial absorbance, using the following approach:

$$Initial A ( λ ) = {C}_{moulded} {A}_{moulded} ( λ ) + {C}_{S a nblasted} {A}_{Sanblas𝔠ed} ( λ )$$

The terms “C_{moulded}” and “C_{sandblasted}” are coefficients using the following values, C_{moulded}=0.225 and C_{Sandblasted}=0.800

Values are calculated with data from both moulded and sandblasted plates, using the following approach:

$$Initial SPF in vitro = \frac{{\int }_{290}^{400} E ( λ ) I ( λ ) d λ}{{\int }_{290}^{400} E ( λ ) I ( λ ) {10}^{− AInitial ( λ )} d λ}$$

### Where:

$$( λ ) = {C}_{m oulded} {A}_{m oulded} ( λ ) + {C}_{S a nblasted} {A}_{Sanblasted} ( λ )$$

E_{λ}: midday mid-summer global irradiance at 40° N
I_{λ}: CIE erythema action spectrum

### EXAMPLES

The following are examples of skin care compositions which are suitable used as sunscreen compositions. The compositions can be prepared, for example, as follows:

In a suitable vessel, the water phase ingredients are combined and heated to 35° C. In a separate suitable vessel, the oil phase ingredients are combined and heated to 65° C. Then the thickener, if included, is added to the oil phase. Next the oil phase ingredients are added to the water phase ingredients, and the resulting emulsion is milled (e.g., with a rotor-stator mill). The emulsion is cooled to 45° C. while stirring. At 45° C., the remaining ingredients, if included, such as neutralized phenylbenzimidazole sulfonic acid and additional neutralizer are added. The product is then cooled with stirring to 30° C. The cooled product is milled again, and then poured into suitable containers.

Detailed compositional breakdown of these compositions is listed as follows (see Tables 1 and 2).

Ex. 1 through Ex. 11 are the examples of the composition disclosed herein, and CEx. A through D are comparative examples. The examples of the composition disclosed herein provide improved UV protection, while providing good skin feel (for example, fresh feel, reduced oily/greasy feel, reduced draggy feel, and/or reduced white residues).

The benefit of the composition disclosed herein, especially improved UV protection can be also understood by the comparison of Ex. 1, 2 and 3 to comparative examples CEx. A-D. In more detail, for example, Ex. 1 which contains 2.5% Particulate UV filer and 3% Wax shows significantly higher SPF improvement than the theoretical SPF improvement (i.e., sum of: the SPF improvement of CEx. B containing 2.5% Particulate UV filer; and the SPF improvement of CEx. D containing 3% Wax). Similarly, Ex. 2 which contains 5% Particulate UV filer and 3% Wax shows significantly higher SPF improvement than the theoretical SPF improvement (i.e., sum of: the SPF improvement of CEx. C containing 5% Particulate UV filer; and the SPF improvement of CEx. D containing 3% Wax)

TABLE 1

| | | CEx. A | CEx. B | CEx. C | CEx. D | Ex. 1 | Ex. 2 | Ex. 3 |
|---|---|---|---|---|---|---|---|---|
| Water | PBSA | 4 | 4 | 4 | 4 | 4 | 4 | 4 |
| soluble UV | | | | | | | | |
| blocking | | | | | | | | |
| active | | | | | | | | |
| Oil soluble | Homosalate | — | — | — | — | — | — | — |
| UV blocking | Octisalate | 4.5 | 4.5 | 4.5 | 4.5 | 4.5 | 4.5 | 4.5 |
| active | Avobenzone | 3 | 3 | 3 | 3 | 3 | 3 | 3 |
| | Octocrylene | 9 | 9 | 9 | 9 | 9 | 9 | 9 |
| Particulate | Methylene Bis- | — | 2.5 | 5 | — | 2.5 | 5 | 5 |
| UV filter | Benzotriazolyl | | | | | | | |
| | Tetramethylbutylphenol | | | | | | | |
| Wax | Sunhancer | — | — | — | 3 | 3 | 3 | 1 |

| Non active base | QS to 100% | | | | | | |
|---|---|---|---|---|---|---|---|
| Total UV % | 20.5 | 23 | 25.5 | 20.5 | 23 | 25.5 | 25.5 |
| SPF (in vitro/SB6 + HD6) | 12.4 | 27.3 | 43.4 | 23.6 | 75.6 | 119 | 77.9 |
| SPF improvement, compared to | n/a | 14.9 | 31 | 11.2 | 63.2 | 106.6 | 65.5 |
| CEx. A | | | | | | | |
| SPF improvement in theory if | — | — | — | — | 26.1 | 42.1 | 31~42.1 |
| additive | | | | | | | |

TABLE 2

| | | Ex. 4 | Ex. 5 | Ex. 6 | Ex. 7 | Ex. 8 | Ex. 9 | Ex. 10 | Ex. 11 |
|---|---|---|---|---|---|---|---|---|---|
| Water soluble | Phenylbenzimidazole | 4 | 3 | 3 | 3 | — | — | 3 | — |
| UV blocking | Sulfonic Acid | | | | | | | | |
| active | | | | | | | | | |
| Water | Tinosorb S lite Aqua | — | — | — | 2 | 3 | 3 | 2 | 2 |
| dispersible UV | | | | | | | | | |
| blocking active | | | | | | | | | |
| Water soluble | Ecamsule (Mexoryl SX) | — | — | — | — | 2 | — | — | 2 |
| UV blocking | | | | | | | | | |
| active | | | | | | | | | |
| Oil soluble UV | Diethylamino | — | — | — | 3 | 3 | 8 | 3 | 8 |
| blocking agent | Hydroxybenzoyl Hexyl | | | | | | | | |
| Particulate UV | Benzoate (Uvinul A | | | | | | | | |
| filter | Plus) | | | | | | | | |
| | Bemotrizinol (Tinosorb | — | — | — | 1 | 3 | 3 | 3 | 3 |
| | S) | | | | | | | | |
| | Ethylhexyl Triazone | — | — | — | — | — | 4 | 3 | 3 |
| | (Uvinul T150) | | | | | | | | |
| | Ethylhexyl | — | — | — | — | — | — | 7.5 | — |
| | Methoxycinnamate | | | | | | | | |
| | Homosalate | 9 | — | — | — | — | — | — | — |
| | Octocrylene | 9 | 9 | 9 | 9 | — | — | — | 2 |
| | Butyl | 3 | 3 | 3 | — | — | — | — | — |
| | Methoxydibenzoylmethane | | | | | | | | |
| | Drometrizole trisiloxane | — | — | — | — | — | — | — | 9 |
| | Methylene Bis- | 5 | 2.5 | — | — | — | 2.5 | 5 | 2.5 |
| | Benzotriazolyl | | | | | | | | |
| | Tetramethylbutylphenol | | | | | | | | |
| | TiO₂ | — | — | 5 | — | — | 2.5 | — | 2.5 |
| | HAA299 | — | — | — | 5 | — | — | — | — |
| | Tinosorb A2B | — | — | — | — | 5 | — | — | — |
| Wax | Sunhancer | 3 | 1 | 1 | 1 | 3 | 1 | 2 | 3 |
| Thickener/ | Acrylates/C10-30 Alkyl | 0.5 | 0.5 | — | 0.5 | — | — | — | 0.5 |
| Emulsifier | Acrylate Crosspolymer | | | | | | | | |
| Thickener | Sodium | — | — | 1.5 | — | 1.5 | 1.5 | 1.5 | — |
| | Polyacryloyldimethyl | | | | | | | | |
| | Taurate | | | | | | | | |
| Emulsifier | PEG-100 Stearate | — | — | — | — | — | — | 0.4 | 0.2 |
| | EMULGADE ® PL | | | | | | | | 0.2 |
| | 68/50 | | | | | | | | |
| | Cetyl Alcohol | — | — | — | — | — | — | 1 | 0.5 |
| | STEARYL ALCOHOL | — | — | — | — | — | — | 1 | 0.5 |
| | Behenyl Alcohol | — | — | — | — | — | — | 1 | 0.5 |
| Low-viscosity | Dimethicone (5 cSt) ¹ | 5 | 5 | 5 | — | — | 2 | — | 2 |
| dimethicone | | | | | | | | | |
| Particulate | Polymethylsilsesquioxane ² | | 5 | | 2.5 | — | 5 | — | |
| Particulate | Tapioca Starch (and) | — | — | — | 2.5 | | 5 | 5 | — |
| | Polymethylsilsesquioxane | | | | | | | | |
| Chelant | Disodium EDTA | — | 0.1 | 0.1 | 0.1 | 0.1 | 0.1 | 0.1 | 0.1 |
| (Water phase) | | | | | | | | | |
| Preservative | 1,2-Hexanediol and 1,2- | 0.8 | 0.8 | 0.8 | 0.4 | 0.8 | 0.8 | 0.8 | 0.8 |
| (Water phase) | Octanediol | | | | | | | | |
| Preservative | Phenoxyethanol | 0.4 | 0.4 | 0.4 | 0.4 | 0.4 | 0.4 | 0.4 | 0.4 |
| (Water phase) | | | | | | | | | |
| Neutralizer | Amino Methyl Propanol | — | — | — | — | — | 1.35 | — | 1.35 |

| | | |
|---|---|---|
| Neutralizer | NaOH | Qs to pH 6-7 |
| | Water | Qs to 100% |

¹ Xiameter ® PMX-200 silicone fluid 5 cs from Dow Corning
² Tospearl CF600 from Momentive

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition, comprising:
a) from about 0.01% to about 50% by weight of the composition, of water-soluble or water-dispersible UV-blocking active;
b) from about 0.1% to about 50% by weight of the composition, of a wax having a melting point of from about 68° C. to about 95° C. and having an average particle size in the composition of from about 100 nm to about 100 microns; and
c) from about 0.1% to about 20% by weight of the composition, of a particulate UV filter.

2. The skin care composition of claim 1, wherein the particulate UV filter is chosen from an organic particulate UV filter, an inorganic particulate UV filter, or mixtures thereof.

3. The skin care composition of claim 2, wherein the particulate UV filter is an organic particulate UV filter.

4. The skin care composition of claim 3, wherein the organic particulate UV filter is Methylene Bis-Benzotriazolyl Tetramethylbutylphenol.

5. The skin care composition of claim 1, comprising from about 1% to about 10% of the particulate UV filter.

6. The skin care composition of claim 5, comprising from about 1% to about 8% of the particulate UV filter.

7. The skin care composition of claim 1, wherein the weight ratio between the water-soluble/dispersible UV-blocking active and the particulate UV filter is from about 3.5:1 to about 1:10.

8. The skin care composition of claim 7, wherein the weight ratio between the water-soluble/dispersible UV-blocking active and the particulate UV filter is from about 3:1 to about 1:5.

9. The skin care composition of claim 8, wherein the weight ratio between the water-soluble/dispersible UV-blocking active and the particulate UV filter is from about 2:1 to about 1:2.

10. The skin care composition according to claim 1, wherein the composition further comprises from about 30% to about 99% of an aqueous carrier.

11. The skin care composition according to claim 10, wherein the composition is in a form of an oil-in-water emulsion.

12. The skin care composition according to claim 1, wherein the water-soluble/dispersible UV-blocking active is chosen from phenylbenzimidazole sulfonic acid, terephthalylidene dicamphor sulfonic acid, disodium phenyl dibenzimidazole tetrasulfonate, salts thereof, or mixtures thereof.

13. The skin care composition according to claim 12, wherein the water-soluble/dispersible UV-blocking active is chosen from of phenylbenzimidazole sulfonic acid salts thereof, or mixtures thereof.

14. The skin care composition according to claim 1, comprising from about 0.5% to about 10%, by weight of the composition, of said water-soluble/dispersible UV-blocking active.

15. The skin care composition according to claim 1, wherein the composition further comprises from about 5% to about 25%, by weight of the composition, of the oil-soluble/dispersible UV-blocking active.

16. The skin care composition according to claim 15, wherein the composition comprises from about 13% to about 23% by weight of the composition, of the oil-soluble/dispersible UV-blocking active.

17. The skin care composition according to claim 15, wherein the weight ratio between the water-soluble/dispersible UV blocking active and the oil-soluble/dispersible UV-blocking active is from about 1:1 to about 1:25.

18. The skin care composition according to claim 17, wherein the weight ratio between the water-soluble/dispersible UV blocking active and the oil-soluble/dispersible UV-blocking active is from about 1:2 to about 1:10.

19. The skin care composition according to claim 18, wherein the weight ratio between the water-soluble/dispersible UV blocking active and the oil-soluble/dispersible UV-blocking active is from about 1:3 to about 1:7.
